(19) United States
(12) Patent Application Publication
(10) Pub. No.: US 2008/0312673 A1
(43) Pub. Date: Dec. 18, 2008

(54) Method and apparatus for CTO crossing

(76) Inventors: Raju R. Viswanathan, St. Louis, MO (US); Gareth T. Munger, St. Louis, MO (US); Heather Drury, St. Louis, MO (US); Christopher D. Minar, New Prague, MN (US)

Correspondence Address: Bryan K. Wheelock;Suite 400, 7700 Bonhomme, St. Louis, MO, 63105, US

(21) Appl. No.: 12/134,150

(22) Filed: Jun. 5, 2008

Related U.S. Application Data
(60) Provisional application No. 60/942,203, filed on Jun. 5, 2007.

(51) Int. Cl. A61B 17/22

(52) U.S. Cl. 606/159

(57) ABSTRACT

A method of treating vessel occlusions including chronic total occlusions (CTO) of the coronary arteries is presented that relies on remote navigation of a remotely steered medical device to the occlusion and controlled application of ablative energy or mechanical push together with real-time local imaging of the vasculature or real-time vessel wall sensing. The combinative use of remote navigation methods and real-time imaging or real-time sensing enables crossing of elongated lesions and CTOs, calcified lesions and CTOs and lesions and CTOs located at vessel branches.

### CROSS-REFERENCE TO RELATED APPLICATIONS

This application claims priority to U.S. Provisional Patent Application Ser. No. 60/942,203, filed Jun. 5, 2007, the entire disclosure of which is incorporated herein.

### FIELD

This invention relates to methods, devices, and systems for occlusion and chronic total occlusion (CTO) crossing therapy and particularly, to the treatment of occlusive coronary artery lesions with a remote navigation system.

### BACKGROUND

Interventional medicine is the collection of medical procedures in which access to the site of treatment is made by navigation through one of the subject's blood vessels, body cavities, or lumens. Interventional medicine technologies have been applied to the manipulation of medical instruments, such as guidewires and catheters, which contact tissues during surgical navigation procedures, making these procedures more precise, repeatable, and less dependent on the device manipulation skills of the physician. Remote navigation of medical devices is a recent technology that has the potential to provide major improvements to minimally invasive medical procedures. Several presently available interventional medical systems for directing the distal end of a medical device use computer-assisted navigation and a display means for providing an image of the medical device within the anatomy. Such systems can display a projection or cross-section image of the medical device being navigated to a target location obtained from an imaging system, such as x-ray fluoroscopy or computed tomography; the surgical navigation being effected through means, such as remote control of the orientation of the device distal end and proximal advance of the medical device.

In a typical minimally invasive intervention, data are collected from a catheter or other interventional device instrumentation that are of significant use in treatment planning, guidance, monitoring, and control. For example, in diagnostic applications right-heart catheterization enables pressure and oxygen saturation measure in the right heart chambers, and helps in the diagnosis of valve abnormalities; left-heart catheterization enables evaluation of mitral and aortic valvular defects and myocardial disease. In electrophysiology applications, electrical signal measurements may be taken at a number of points within the cardiac cavities to map cardiac activity and determine the source of arrhythmias, fibrillations, and other disorders of the cardiac rhythm. For angioplasty applications, a number of interventional tools have been developed that are suitable for the treatment of vessel occlusions: guidewires and interventional wires may be proximally advanced and rotated to perform surgical removal of the inner layer of an artery when thickened and atheromatous or occluded by intimal plaque (endarterectomy). Reliable systems have evolved for establishing arterial access, controlling bleeding, and maneuvering catheters and catheter-based devices through the arterial tree to the treatment site. Systems for coronary arteries are similar, but the smaller size (3 to 5 mm proximally) and greater tortuosity of the coronaries require smaller and more flexible devices.

The primary objective of angioplasty is to re-establish a stable lumen with a diameter similar to that of the normal artery. This goal may be achieved by using a variety of interventional devices, including angioplasty balloons, lasers, rotoblators, and stents. In recent years, the introduction of specially designed catheters comprising strong inflatable balloons at or near their distal end, as well as along the length of the device, has greatly changed the field of minimally invasive cardiovascular surgery. The balloons are used for percutaneous transluminal coronary angioplasty (PTCA) to dilate a partially obstructed artery and restore blood flow to the myocardium; balloon catheters are also used to treat heart valve stenosis. Although there are risks associated with the procedure, such as tearing or embolization, the technique may be applied to several coronary arteries with excellent results, and may be repeated if necessary. All new developments in the field of percutaneous coronary intervention (PCI) have been targeted to do one or more of the following: i) reduce treatment risk, ii) reduce the occurrence of restenosis; and iii) allow more complex cases to be treated via minimally invasive techniques. In particular, a number of new devices and associated techniques have been developed in an attempt to increase the chronic total occlusion (CTO) treatment success rate; up to now however, the use of devices to increase the success rate in angioplasty of CTO has been accompanied by an increase in complication rate.

Restenosis is the major limitation of angioplasty. Restenosis is a complex process comprising three separate mechanisms: early recoil, neo-intimal hyperplasia, and late contraction (negative remodeling). Arterial plaque begins in the intima by deposits of fatty debris from blood. As the disease progresses, lipids accumulate in the intima to form yellow fatty streaks. A fibrous plaque begins to form. Eventually a complex lesion develops as the core of the fibrous atherosclerotic plaque necroses, calcifies, and hemorrhages. Angioplasty leads to a fracture of the atherosclerotic plaque, the intima, and sometimes fractures extending into the media. Immediately following balloon angioplasty, the elastic medial vessel layer contracts (early recoil). Over weeks, neo-intimal cell proliferation results in new tissue growth occupying the cracks and tears in the vessel wall, new tissue becomes less cellular and the healing sites begin to resemble a fibrous plaque (neo-intimal hyperplasia). In most subjects, the lumen enlarging effect of angioplasty outweighs the lumen-narrowing effect of neo-intimal hyperplasia. However, in about 40% of subjects, neo-intimal hyperplasia is excessive, and results in clinically symptomatic restenosis within three to six months. This effect is compounded by late arterial contraction (negative remodeling).

Angioplasty enlarges the lumen by stretching and splitting the wall; in some cases this is made impossible by lesions with a lumen too small for the balloon to cross, or by heavy calcification of the arterial wall, making it too tough and inelastic to split or stretch. In these cases, it may be necessary to remove tissue by cutting (atherectomy device), abrading (rotoblator), or vaporizing (laser). Because the risk of arterial wall perforation is clearly much higher with these methods, they are usually not applied aggressively to achieve the desired final lumen size; rather, they are used to initially “debulk” the lesion, and then followed by balloon angioplasty and/or stent placement.

Stent placement following angioplasty effectively repairs vessel wall dissections, prevents tissue flaps from protruding in the lumen, resists elastic recoil, and minimizes loss of lumen diameter due to negative remodeling. Stents by themselves however do not eliminate restenosis, as they appear to stimulate proliferation. Restenosis is best addressed by placing a drug eluting stent in the balloon-treated lesion or by irradiating the treated vessel segment by brachytherapy. These restenosis preventive treatments have made a profound impact on the mid and long-term viability of narrow vessel and CTO disease treatment.

Chronic total occlusions are present in about 30% of the 1.5 million diagnostic angiograms performed every year in the United States. However, up to now minimally invasive treatment of CTOs has been difficult, and only about 10% of angioplasty interventions are directed at CTO therapy; indeed CTO presence often precludes treatment by coronary percutaneous intervention and remains a major reason for referral for coronary artery bypass graft surgery (CABG). Treatment success rate is typically in the 60%-85% range; yet a significant number of CTO lesions are left untreated because of uncertainties regarding procedural success and long term benefit. Procedural shortcomings and complications include failure to cross with the guidewire or balloon, failure to dilate the lesion, failure to deploy a stent, and myocardial infarction. Additional risks include distal perforation and/or arterial dissection and associated complications such as haemo-pericardium, cardiac tamponade, and death, and the possible need for prompt pericardiocentesis and reversal of anticoagulation and/or emergency CABG surgery; and embolization. In general, attempts at treating CTOs with current technologies are not recommended when: i) the CTO presents an extended blockage, for example greater than 15 mm; ii) the CTO is heavily calcified; iii) there is poor distal vessel visualization, and the introduction of a retrograde wire is difficult or there is no prospect for retrograde access; iv) the CTO has been present for an extended period of time, for example, more than three months; v) the lesion presents with irregular contours, in eccentric anatomy, or with antegrade collaterals; or vi) thrombus is present. However, recent clinical data indicate that successful CTO treatment and artery opening induce significant long-term morbidity and mortality advantages, including reduction or elimination of angina pectoris symptoms, improved left ventricular function and ejection fraction, reduced myocardial infarction and lower incidence of cardiac death. Clinical data support aggressive attempts to open chronically occluded vessels when favorable treatment factors exist, such as the presence of a tapered stump at a branch, pre- or post-branch occi, absence of bridging collateral vessels, and presence of a functional occlusion. The development and availability of new techniques capable of safely and effectively treat the most difficult cases would most likely induce significantly favorable clinical outcomes.

New CTO techniques developed recently include mechanical and ablative approaches. Mechanical technologies include the use of polymer coated or tapered wires, low profile balloons, blunt micro-dissection to attempt to gently separate atherosclerotic plaques in various tissue planes to create a passage through the CTO by using the elastic properties of adventitia versus the inelastic properties of fibro-calcific plaque to create fracture planes. Ablative technologies include the use of excimer lasers, ultrasound or vibrational techniques (activated guidewire angioplasty) to induce cavitation, as for example, by delivering controlled acoustic energy along the active section of a thin wire; the infusion of collagenase at the CTO through a thin catheter to soften the occlusion and enable wire crossing; and the recent development of radio-frequency (RF) approaches. Stent deployment, if the artery can be opened, has been shown to improve outcome. In particular, balloon angioplasty data indicate that the need for emergency CABG has fallen since stenting has become routine. Stiff guidewires, while providing increased pushability and torque response are more likely to create false channels, dissection and perforation. Hydrophilic guidewires have a polymer coating that becomes very slippery once moistened, which reduces thrombus adhesion and facilitates the advancement of the wire within the occlusion.

An excimer laser wire was developed to attempt crossing CTOs in the event of a failure with any guidewire. As the results of the TOTAL trial (Total occlusion trial with angioplasty by using laser guidewire) indicate, although laser guidewire technology was safe, the increase in crossing success did not reach statistical significance. The most frequent reasons for laser guidewire failure were false route tracking and misalignment, while the most common reason for failure in the mechanical wire group was absence of wire progression. Accordingly, increasing lesion penetration power by itself is not sufficient to lead to significant favorable clinical outcomes.

U.S. Pat. No. 6,394,956 issued to Chandrasekaran et al. and assigned to Scimed Life Systems, Inc. (now part of Boston Scientific), discloses a combination catheter, including an intravascular ultrasound (IVUS) device and an RF ablation electrode. RF ablation proceeds by depositing energy to locally raise the tissue temperature to fulguration. RF power for inter-arterial lesion ablation is typically delivered in pulses to allow heat dissipation and avoid damaging adjacent healthy tissues. In one embodiment, pulses are delivered at a rate of about 10 Hz to about 10 kHz. Each ablative pulse is typically delivered with a frequency of about 200 kHz to about 2 MHz, although a typical electrosurgical power generator might operate within a frequency range from about 200 kHz to about 35 MHz. The RF circuit voltage may be as high as 1 kV, and delivered power in the range 1 to 50 watts depending on the application. Ultrasound imaging provides feedback regarding the relative position of the device distal end and vessel tissues, so as to reduce the risks associated with RF energy delivery to the vessel walls. Various RF electrode configurations are possible, including protruding hemispherical shape, roughened protruding hemispherical, concave electrode surface, or extendable intermeshed wires enabling variable electrode diameter. Although Pat. No. 6,394,956 describes a mechanical pull-wire navigation system, it does not teach nor suggest the combinative use of other navigation means, such as magnetic or electrostrictive actuation with RF lesion ablation. Accordingly, the navigation limitations associated with the use of a mechanical pull-wire system, including limited distal end steering, are not addressed nor solutions suggested in U.S. Pat. No. 6,394,956. Despite its value in visualizing true lumen dimensions, vessel wall composition, and controlling the intervention, IVUS for now remains a niche product used by a limited, albeit increasing, number of physicians.

Other recently developed techniques, include the use of optical coherence reflectometry (OCR) and optical coherence tomography (OCT) for the characterization and direct visualization of tissues. In at least one application, OCR has been used to provide as binary signal information that the distance from the device to the vessel wall is less than a given threshold. In one embodiment, OCR uses an optic fiber placed through a support catheter or guidewire to illuminate tissue with a low coherence light; reflected and scattered light patterns are detected and analyzed to differentiate between plaque and normal arterial wall; it has been shown, that light scattering intensity increases when scattering originates from a healthy arterial wall as compared to arterial occlusive materials. U.S. Pat. No. 6,852,109 issued to Winston and Neet and assigned to IntraLuminal Therapeutics, Inc. (now part of Kinsey Nash Corporation), describes a guidewire assembly, including a guidewire electrically connected to an RF power generator and an optical fiber connected to an optical reflectometer. The assembly may comprise either a unipolar or bipolar RF electrode(s). RF power may be gated to an ECG signal to ensure that power is not delivered during the ECG S-T segment, as the heart is most sensitive to electrical signals during this period; indeed it is known that RF pulse triggering may induce cardiac systole. Also, RF sub-system design may include a control to ensure that RF power is delivered only when the RF electrode is in tissue contact. Although the combination of RF ablation capability with OCR characterization helps to reduce adverse events, such as arterial perforation or dissection, the methods and devices disclosed in U.S. Pat. No. 6,852,109 do not teach nor suggest how to improve on the state-of-the-art for device distal end navigation, localization, and positioning with respect to the vessel walls and lesions. In clinical trials utilizing the technology described in U.S. Pat. No. 6,852,109, limited steerability (in particular within the lesions) remained a problem.

Additional tissue and arterial plaque characterization techniques have been developed and are being investigated for application to the treatment of the coronary arteries. U.S. Pat. No. 6,949,072 issued to Furnish S, et al. and assigned to InfraRedX, Inc., discloses the use of near-infrared (NIR) diffusion reflectance spectrometry together with intra-vascular ultrasound (IVUS) transducer for the characterization of tissues and the detection of “vulnerable plaque.” Vulnerable plaque, assumed to be mostly liquid rich, as opposed to fibrous plaque, is a major cause of heart attack through the mechanism of plaque rupture and subsequent thrombus formation and artery blockage. The probe of Pat. No. 6,949,072 is inserted over a pre-navigated guidewire, and does not teach the use of remote steering or navigation means, such as magnetic or electrostrictive actuation, with RF lesion ablation and/or optical or ultrasound imaging or characterization. Accordingly the navigation limitations associated with the use of guidewires, including limited distal end steering, are not addressed nor solutions suggested in U.S. Pat. No. 6,949,072.

Additionally, bifurcation CTO lesions in small vessels are particularly difficult to treat. Identification of the best approach to bifurcation disease remains unresolved. It is debatable whether PCO using current technology, is the treatment of choice for such cases because of technical problems and high incidence of acute and chronic events.

### SUMMARY

Three technology requirements for the crossing of most challenging CTOs are addressed individually and collectively by various embodiments the present invention: increased lesion penetration power as compared to guidewires without the need for large proximal force application; tissue characterization and differentiation capability, possibly including direct visualization/imaging, to reduce the likelihood of adverse events; and steerability of the device distal end to keep the ablation device oriented along the main local vessel axis, therefore, enabling ablative power application or mechanical crossing. Embodiments of the present invention provide methods of performing CTO ablation therapy by guiding a wire, catheter or interventional device to the occlusion, characterizing or visualizing the tissues in the vicinity of the device distal end, orienting a crossing wire, possibly including an RF ablation electrode, applying either mechanical push forces or RF power or other ablative means to the occlusion through the wire or catheter, and iteratively navigating the wire or catheter through the lesion, characterizing tissues, and applying either mechanical push or RF or other ablative power to create an opening therethrough. Further, some embodiments of the invention provide methods of navigating a crossing therapy device by magnetic navigation means, mechanical navigation means, electrostrictive navigation means, or combination thereof. Use of magnetic navigation in combination with RF ablation enables the use of thinner, more maneuverable wires as pushability and torque transfer requirements decrease. Likewise, the use of a magnetically navigated guidewire capable of applying suitable levels of mechanical push force within the lesion holds the potential for easier methods of therapy delivery. Current CTO intervention failures stem from inability to cross the occlusion with a guidewire, inability to access the lesion due to tortuous vascular anatomy, or from lesion restenosis or reocclusion. Restenosis is a particularly significant problem for small (<3 mm) vessel disease. The ability to cross the lesion with a thinner wire enables advancement of a lower profile balloon catheter, and thus, the treatment of smaller arteries, including the capability of placing stents and drug-eluting stents (or the use of brachytherapy) in smaller arteries. Stents address both elastic vessel recoil and negative remodeling; drugs eluting stents have a robust effect on tissue growth, and very significantly, bring down the rate of restenosis. Accordingly, both CTO treatment failure modes are addressed by magnetic navigation of a CTO crossing device, whether mechanical or ablative, as described below.

### BRIEF DESCRIPTION OF THE DRAWINGS

FIG. 1-A shows a subject positioned in a projection imaging system for an interventional procedure, such as percutaneous coronary intervention (PCI) and therapy using a controlled minimally invasive modality, such as RF ablation;

FIG. 1-B illustrates an interventional device distal end being in occlusion contact within a theater of intervention, such as an artery;

FIG. 2 presents a workflow chart for a method of coronary intervention and chronic total occlusion therapy, according to some embodiments of the present invention;

FIG. 3 schematically shows a radio-frequency interventional device creating a crossing through a vessel CTO;

FIG. 4 schematically illustrates the use of an interventional device according to the principles of the present invention for the treatment of a CTO at a vessel branch;

FIG. 5 describes terminology and vessel topology used in the remainder of the disclosure;

FIG. 6 presents a method of imaging orientation registration using vessel contours;

FIG. 7 shows schematically a method of imaging orientation registration using wire movements;

FIG. 8 illustrates a method of imaging orientation registration using wire, imaging catheter, and fluoroscopy;

FIG. 9 schematically demonstrates application of the methods and devices of some embodiments of the present invention to CTO crossing with a forward-looking imaging catheter;

FIG. 10 illustrates the use of vessel wall sensing technology to the navigation of a therapeutic device and lesion crossing;

FIG. 11 presents the use of side-looking imaging to progress through a CTO;

FIG. 12 shows the use of side-looking imaging when treating a branch lesion;

FIG. 13 illustrates energy application gated to the subject's ECG;

FIG. 14 schematically demonstrates the use of imaging and real-time location feedback to the navigation of complex vessel anatomy;

FIG. 15 further illustrates the location feedback information and its use in navigation;

FIG. 16 presents a dynamic x-ray imaging sequence of use in mapping and navigating leg anatomy; and

FIG. 17 shows one embodiment of an RF and imaging device.

Corresponding reference numerals indicate corresponding points throughout the several views of the drawings.

### DETAILED DESCRIPTION

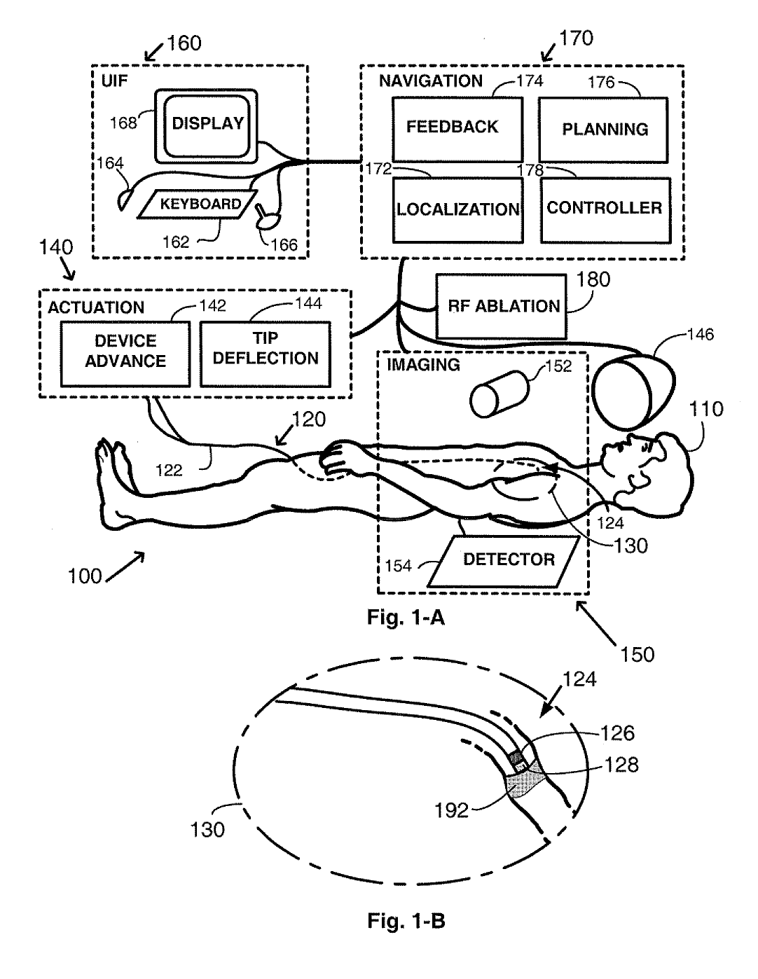

As illustrated in FIG. 1, a subject 110 is positioned within an interventional system, 100. An elongated navigable medical device 120 having a proximal end 122 and a distal end 124 is provided for use in the interventional system 100. FIG. 1-A shows the medical device inserted into a blood vessel of the subject and navigated to an intervention volume 130. A means of applying force and orienting the device distal end 124 is provided, as illustrated by actuation block 140, comprising a device advance/retraction component 142 and a tip deflection component 144. The tip deflection means may be remotely actuated, with the means of remote actuation comprising one of (i) a mechanical pull-wire system; (ii) a hydraulic or pneumatic system; (iii) an electrostrictive system; (iv) a magnetically actuated system; or (v) other navigation system, as known in the art. For illustration, in magnetic navigation a magnetic field externally generated by a magnet(s) assembly 146 orients a small magnet located at the device distal end (126, FIG. 1-B). Real time information is provided to the physician by an imaging sub-system 150, for example, an x-ray imaging chain comprising an x-ray tube 152 and an x-ray detector 154, and also possibly by use of a three-dimensional device localization sub-system, such as a set of electromagnetic wave receivers located at the device distal end (not shown) and associated external electromagnetic wave emitters (not shown), or other localization device with similar effect. The physician provides inputs to the navigation system through a user interface (UIF) sub-system 160 comprising user interfaces devices, such as a display 168, a keyboard 162, mouse 164, joystick 166, and similar input devices. Display 168 also shows real-time image information acquired by the imaging system 150 and the three-dimensional localization system. UIF sub-system 160 relays inputs from the user to a navigation sub-system 170 comprising a 3D localization block 172, a feedback block 174, a planning block 176, and a controller 178. Navigation sequences are determined by the planning block 176 based on inputs from the user, pre-operative data, localization data processed by localization block 172 and real-time imaging and feedback data processed by feedback block 174; the navigation sequence instructions are then sent to the controller 178 which actuates the device through actuation block 140 to effect device advance and tip deflection. Other navigation sensors might include an ultrasound device or other device appropriate for the determination of distance from the device tip to the tissues or for tissue characterization (not shown). Further device tip feedback data may include relative tip and tissue positions information provided by an imaging system, predictive device modeling, or device localization system. In the application to occlusion ablation, additional feedback may be provided by an IVUS device, an optical coherence reflectometry device, an optical coherence tomography device, or similar device that allows intravascular and vascular characterization to separate plaque or fibrous lesion from vascular wall (not shown). In closed loop implementation, the navigation sub-system 170 automatically provides input commands to the device advance and tip orientation actuation components based on feedback data and previously provided input instructions; in semi-closed loop implementations, the physician fine-tunes the navigation control, based in part upon displayed and other feedback data, such as haptic force feedback information. Control commands and feedback data may be communicated from the user interface 160 and navigation sub-system 170 to the device and from the device back to navigation sub-system 170 (feedback) through cables or other means, such as wireless communications and interfaces, As known in the art, system 100 comprises an electromechanical device advancer 142, capable of precise device advance and retraction based on corresponding control commands. In RF therapy applications, an RF component 180 may collect temperature data measured at the device tip 124 by electrode 128 in contact with tissue, FIG. 1-B. The RF-capable device is advanced into contact with the occlusion 192, RF power is applied, and the device is navigated through the occlusion; iteration of the above sequence, under real-time imaging, tissue characterization, temperature and localization control, enables crossing the CTO. RF electrode design depends on a number of parameters, such as target vessel size, expected occlusive materials to be ablated and other parameters, as known in the art.

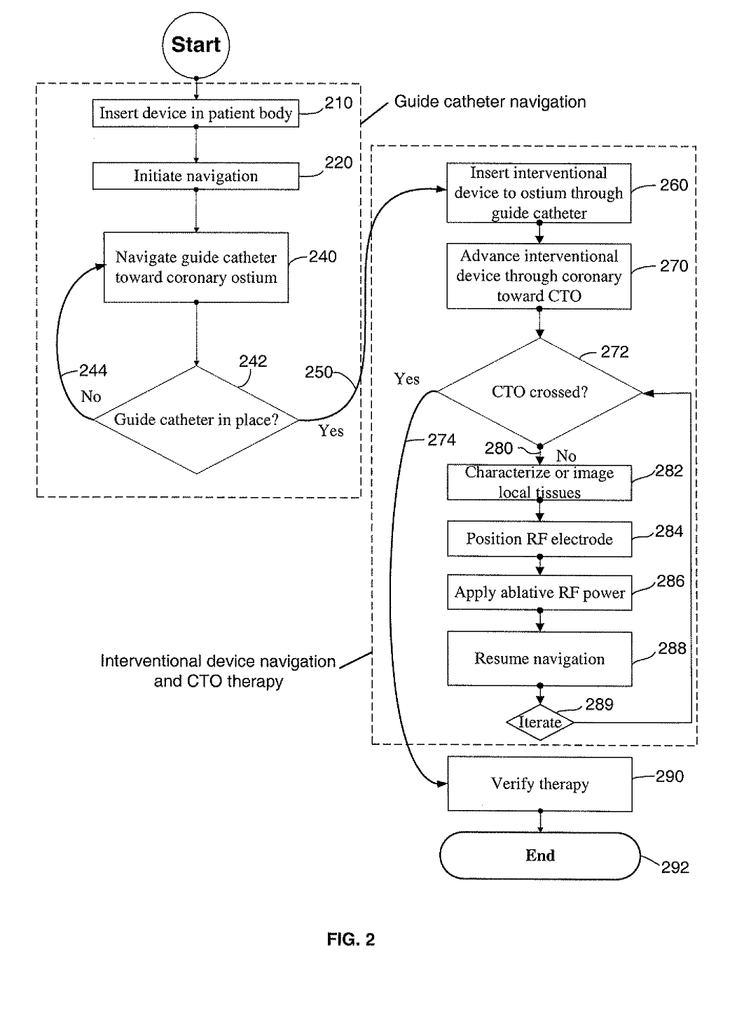

Referring now to FIG. 2, a flow-chart for a method of CTO ablation therapy according to the principles of the present invention is presented, as applied to the treatment of a coronary artery occlusion with interventional device magnetic navigation. A guide catheter for the interventional device is inserted into a vessel of subject, 210, and magnetic navigation is initiated, 220. The guide catheter is navigated towards the coronary ostium 240 and the method iterated through steps 242 and 244 till the distal end is firmly in place, 250. Then an RF-capable interventional device, such as an RF wire or catheter specifically designed for the delivery of RF ablative energy to vessel occlusions, is inserted to the ostium through the guide catheter, 260, and navigated beyond the ostium through the coronary toward the CTO, 270. At decision block 272, if the CTO was crossed by advancing the interventional device, 274, the coronary blood flow and pressures may be measured or other steps taken, to verify the therapy, 290. Otherwise, step 280, local tissues in the vicinity of the RF electrode are characterized, for example, by use of IVUS or OCR, 282; or alternatively a real-time image of the local tissue is acquired, 282; and the device distal end and RF electrode are positioned in contact with the lesion and oriented with respect to the local vessel and occlusion anatomy to ensure lesion ablation while respecting the integrity of the arterial wall, 284, ablative RF power is applied under temperature and localization control, 286, the interventional device is navigated through the lesion opening just created, 288, and the method is iterated 289 till the CTO is crossed, 274. The real-time image can be acquired either as a direct forward image (imaging a region in front of the distal tip of the catheter) or as a side-looking image (imaging a region around and transverse to the distal tip of the catheter). Finally, the therapy is verified in step 290 and the method terminates 292. Alternatively to IVUS or OCR, other methods, such as optical coherence tomography may be used, as known in the art.

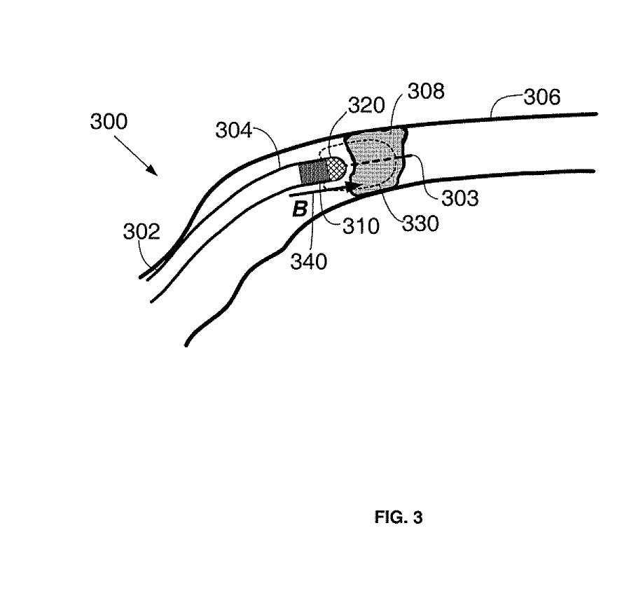

FIG. 3 schematically presents 300, a magnetically navigated RF interventional device 302 being navigated through an artery 306 to contact a CTO occlusion 308. The distal end 304 of the device comprises a magnet 310 sufficient for magnetic navigation in an applied field of about 0.1 Tesla, and preferably no more than about 0.08 Tesla, and preferably no more than about 0.06 Tesla. The device tip comprises an RF electrode 320 for application of ablative power to a lesion volume 330. During the intervention, a magnetic field B 340 externally generated by sub-system 146 is applied to align the device distal end 304 with the local vessel axis 303; pressure is exerted to the lesion by proximally controlling the device advance and RF power is applied, typically in a sequence of pulses. Various RF electrode designs for CTO therapy are possible, including a mono-polar design, wherein RF power is returned to the RF generator through a patch electrode applied to the subject's skin, the electrode patch typically being positioned on the subject's back. The volume 330 through which a given amount of power is deposited in the lesion is dependent upon RF electrode design parameters and local tissue characteristics, as known in the art. Iterative application of ablative power and device navigation under real-time temperature, localization and imaging control enables crossing most CTOs. In particular, use of RF ablative power enables treatment of elongated CTOs, as well as crossing densely calcified lesions. It is emphasized that by design of the interventional system and device, maneuverability of the device distal end, in most cases enables positioning and orientation of the RF electrode, such that only diseased tissue at a safe distance margin from the vessel wall are ablated.

While the use of a RF-capable device was described in the above, it is also possible to work with a device, such as a guidewire that is capable of applying a suitably strong mechanical push to cross the occlusion in a series of small steps involving iterative application of steering/tip reorientation and mechanical pushing of the wire to burrow into the lesion, possibly accompanied by twirling the wire about its axis to release/reduce friction.

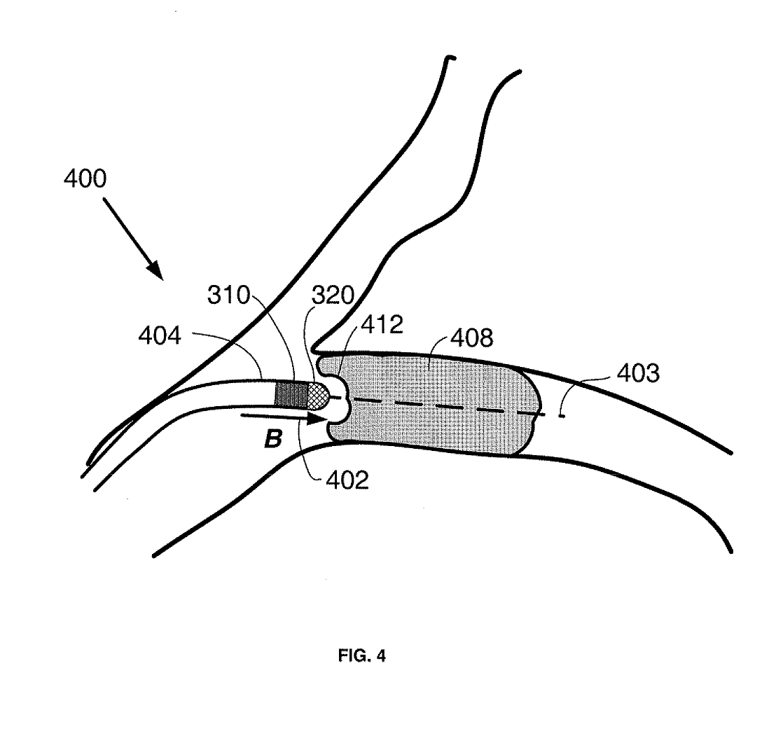

Referring now to FIG. 4, one embodiment of the method of the present invention is applied to the treatment of a branch CTO, 400. Branch CTOs are among the most difficult cases of narrow artery disease to treat with current state-of-the-art technologies, The relative length of the lesion (as for example longer than 15 mm) makes it very unlikely to be successfully crossed by conventional approaches using thin tapered mechanical guidewires. When attempting CTO crossing by advancing a thin tapered wire, the geometry of the vessels and the presence of a lesion at a vessel branch often lead to device prolapse into the adjacent vessel. Alternatively, presence of the lesion at the branch without a tapered stump would likely lead to distal wire sliding into the adjacent, non-occluded, branch, and failure to perform therapy. When using magnetic navigation, an externally generated B field 402 is applied to the device distal end 404 comprising a small magnet 310, to align the device with the local vessel axis 403. RF power is applied to electrode 320 when the device tip is in contact with the lesion 408 at surface 412. Iterative application of ablative power and magnetic navigation and device advance enables lesion ablation along the local vessel axis 403 and successful CTO crossing. The use of ablative RF power in combination with magnetic navigation enables creation of a passage way through the lesion with minimum proximal advance force being applied, thereby, avoiding distal device buckling and prolapse, and avoiding distal end slippage away from the lesion and into the parent branch.

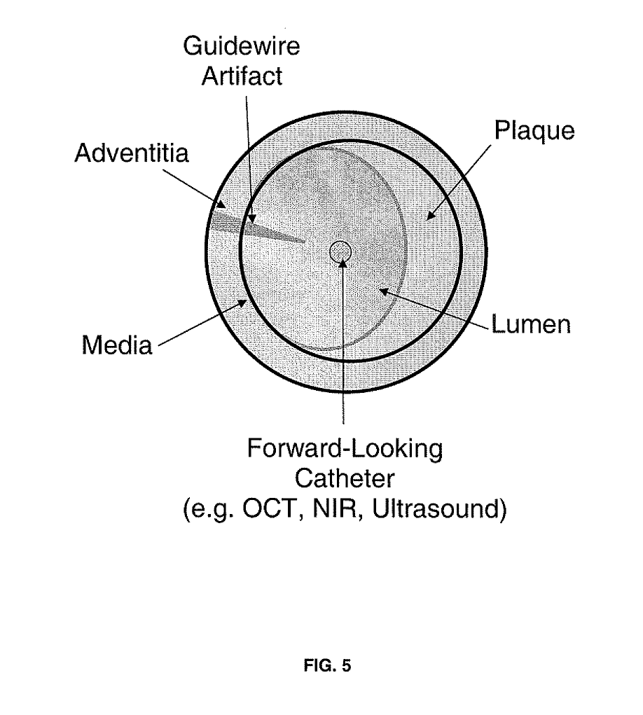

FIG. 5 illustrates vessel anatomy comprising several layers. The outermost layer or external vessel coat is the adventitia, consisting mostly of fibroelastic tissue; the middle layer or vessel coat, the media, consists chiefly of circularly arranged muscle fibers; and the innermost coat, or intima, or the larger blood vessels consists of an endothelial lining backed by a layer of connective tissue and a layer of elastic tissue. The vessel defines a lumen for the passage of blood. A common disease process is the formation of plaque, particularly along arterial vessels. As illustration, the basis of coronary artery disease is the slow development of areas of thickening in the arteries, called atherosclerotic plaques, or atheromatous lesions. Such lesions or plaques often develop early in life, progressing over a period of many years with phases of quiescence or even regression interspersed with periods of progression. Coronary lesions are found in virtually all adults in the industrialized world. Although most people who have these lesions will never develop signs or symptoms of heart disease, in others the lesions intrude into the lumen of the coronary arteries, progressively impeding blood flow to the myocardium and leading to the clinical syndromes of coronary heart disease. Two major factors determine the growth of atheromatous lesions. One is the accumulation of cholesterol at the areas where the thickening occurs and the other is the incorporation of minute clots, or thrombi, into the endothelial surface of the artery. Accumulation of cholesterol in atherosclerotic lesions is related to the concentration of cholesterol-carrying lipoproteins in the blood that flows through the coronary arteries. Elevation of the concentration of these lipoproteins is primarily determined by genetic factors, but can also be influenced by environmental factors, such as a high-fat diet. Under most conditions the incorporation of cholesterol-rich lipoproteins is the predominant factor in determining whether or not plaques progressively develop. Then, the endothelial injury that results leads to the involvement of two cell types circulating in the blood-platelets and monocytes. Platelets adhere to areas of endothelial injury and to themselves. They trap fibrinogen, a plasma protein, leading to the development of platelet-fibrinogen thrombi. Platelets, monocytes, and other elements of the blood release hormones, called growth factors that stimulate proliferation of muscle cells in arteries. Atherosclerotic lesions are focal and their distribution is determined by the interrelation of hemodynamic physical forces such, as blood pressure, blood flow, and turbulence within the lumen. These lead to physical forces of parallel strain, or shear, on the endothelial lining, giving rise to areas of relatively positive and negative pressure. These hemodynamic forces are particularly important in the system of coronary arteries, where there are unique pressure relationships. The flow of blood through the coronary system into the heart muscle takes place during diastole (phase of ventricular relaxation) and virtually not at all during systole (the phase of ventricular contraction). During systole, the external pressure on coronary arterioles is such that blood cannot flow forward. The external pressure exerted by the contracting myocardium on coronary arteries also influences the distribution of atheromatous obstructive lesions.

FIG. 5 also illustrates the progression of a catheter comprising a forward or side-looking imaging or tissue characterization means, such as ultrasound, NIR, or OCT. A guidewire is present on the left side of the catheter, and projects in the acquired image a shadow cone that extends from the guidewire to the vessel wall (“guidewire artifact”).

Since the orientation of the image produced by the imaging catheter, whether side-looking or forward-looking, is not fixed, in general registration of this real-time image with the remote navigation system is desirable so that user interaction and control of the device can be made more intuitive. Any of at least the 3 following methods can be used to register/align the image produced by the imaging catheter with three dimensional anatomy.

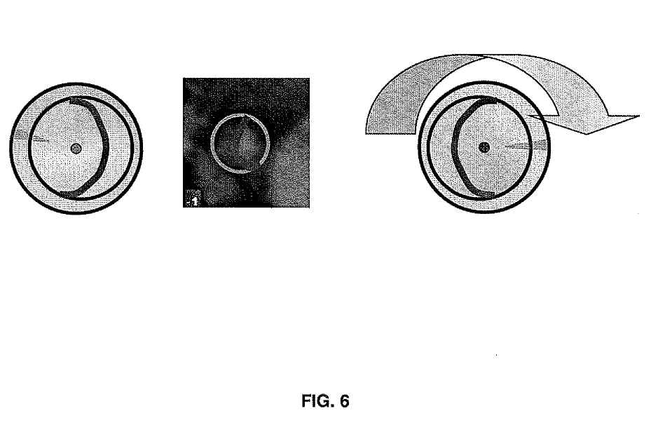

Contour-based registration to pre-operative 3D data proceeds by marking the contour of plaque or other landmark on the real-time images; marking the plaque contour on pre-operative three-dimensional (3D) data; and reorienting the 3D preoperative views to correspond to the real-time image. This process is now further illustrated. When pre-operative 3D image data (such as CT or MR) of the vasculature is available, it can be sliced in a direction orthogonal to the local vessel centerline. The pre-operative vasculature can be registered to X-ray coordinates by marking on a suitable set of points, as is illustrated in FIG. 6.

The slices above, when taken in the region proximal to the Chronic Total Occlusion, can usually show a contour of the putative vessel boundary/lumen, or portions thereof. The slices can be displayed in some canonical fashion, analogous to the bulls-eye on the Stereotaxis magnetic navigation User Interface, Navigant™, such that certain canonical directions (Superior etc.) are in anatomically sensible contexts (e.g., Superior is always “up” in the display). The real-time image obtained from the imaging catheter can also show the vessel/lumen boundary contour.

An edge shape-matching algorithm can find the rotated real-time image whose vessel boundary contour best matches the boundary contour obtained from the 3D pre-operative data set at the same location along the vessel.

Once such a rotation is found, it is consistently applied in the display of the real-time image, so that the real-time image is now always displayed in canonical fashion. As before, in one preferred embodiment, the real-time image itself could be used, after it has been registered, in 6 manner analogous to the bulls-eye display for device steering/navigation purposes.

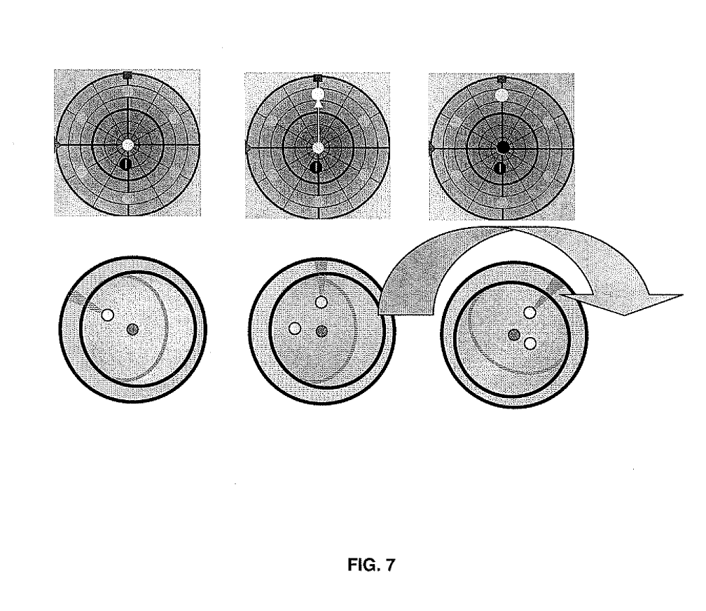

The process of local actuation control, illustrated in FIG. 7, comprises marking the tip of the guidewire on the real-time image (yellow circle), orienting the wire in a known direction, for example, superior marking the new or second position of the wire on the real-time image, and automatically re-orienting the real-time image to align with respect to the direction given from the two identified wire locations. This process is now described in more detail. In the case of one preferred embodiment where the remote navigation system is a magnetic navigation system that steers the (magnet-tipped) crossing wire, the movement direction of the crossing wire that results upon an application of a change in magnetic field direction can be used for registration. First, the crossing wire is positioned proximal to the occlusion (so that the tip can move freely within the confines of the vessel it is in) and is centered by applying an appropriate field direction, for instance, by using the vessel navigation capability on the magnetic navigation system that generates a magnetic field that causes the wire tip to be substantially aligned with the local vessel tangent direction. In the real-time image, the wire will either be seen (if it is not in the blind spot of the imaging catheter) or not seen (if it is in the blind spot of the imaging catheter). In either case, the approximate location of the wire in the real-time image can be marked (for instance with a mouse-click, immediately after an “Align” button is pressed).

Next, the bulls-eye display on the magnetic navigation system User Interface (UI) is used to represent the local cross-sectional plane by centering it at the current field direction. The bulls-eye display includes canonical direction markers (representing, for instance, Superior and Right Lateral directions) as a reference. One of these reference markers can be used to define a field change that represents a change in field in that direction (say towards Superior) by suitably clicking on the bulls-eye display. The wire will generally move in approximately the same direction in three dimensional space. Within the real-time image, the wire will now appear at a different location. This new location in the real-time image is marked by the user, followed for instance by the press of a “Done” button.

The system uses the information about the old and new wire locations to then effect a rotation of the displayed image, so that the movement direction of the wire (from the old to the new location in the real-time image) is aligned with the change in field direction (towards Superior); now the rotated displayed image is aligned with the bulls-eye display, and changes in field direction will produce corresponding intuitive changes in wire position.

In one preferred embodiment, the real-time image itself could be used, after registration, in a manner analogous to the bulls-eye display for device steering/navigation purposes.

In other embodiments, the remote navigation system could employ robotically/mechanically driven guide catheters, or other actuation methods, such as electrostriction, pneumatic or hydraulic control.

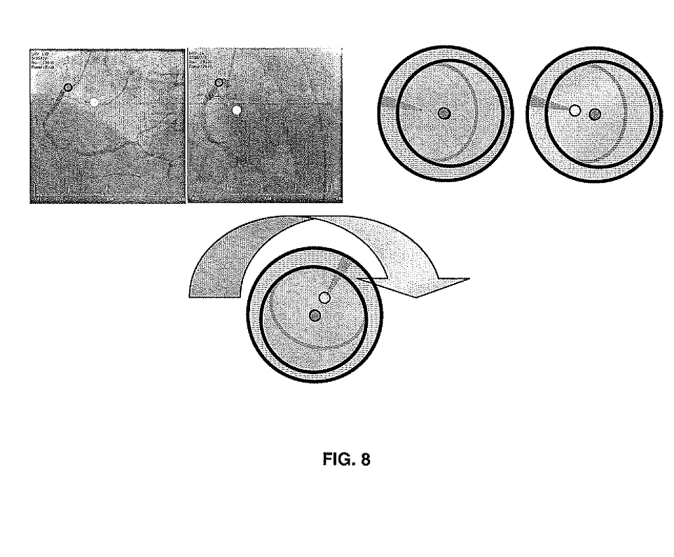

Imaging orientation registration can also be achieved using the wire, intra-vascular imaging device, and x-ray image data from a fluoroscopy system. This is described in FIG. 8, where the steps illustrated comprise marking the catheter tip on two x-ray images, marking the tip of the guidewire on two x-ray images, marking the wire and catheter tips on the real-time image(s), and reorienting to achieve registration. This process is now described in more details. In this scheme, the imaging catheter and crossing wire are positioned with sufficient transverse (˜1-2 mm) separation between each other's tips, proximal to the occlusion, such that each device tip can be identified and marked on each of two X-ray views. Preferably, the X-ray views are separated by an angular separation of at least 40 degrees between them.

Now three dimensional coordinates of the two device tips can be obtained from this information, in Fluoro coordinates (and thus, in remote navigation system coordinates).

Next the user marks the wire location in the real-time image; the imaging catheter is at the center of this image. Thus, the catheter-to-wire vector vᵣ in the real-time image is known.

It is assumed that the centerline of the vessel (in three dimensions) is known, from either marking on multiple X-ray views, or image processing-based vessel edge detection methods with contrast-filled vessels, or from registered pre-operative 3D data (for instance CT or MR data). In particular, this means the local tangent t to the vessel centerline is known at the location of the wire tip. The three dimensional catheter-to-wire vector v is known, since the user has marked their tip locations; the system then finds a rotation of v about t to a new vector v′ such that the dot product of v′ with the Superior direction s is maximal. Let the corresponding rotation angle be θ.

Next the system rotates the real-time image by an angle φ, such that the rotated version of the catheter-to-wire vector vᵣ now makes an angle of θ with respect to the vertical in this image. Now the real-time image has been aligned, in effect, with the bulls-eye view where the Superior direction is indicated at the top (again, such that the vertical direction has maximal dot product with the Superior direction).

As before, in one preferred embodiment, the real-time image itself could be used, after it has been registered, in a manner analogous to the bulls-eye display for device steering/navigation purposes.

As described in the Background, it is clear that the capability to quickly and repeatedly navigate devices to a treatment site through complex anatomy is essential to progress in the clinical outcomes of many therapies, including CTO treatment.

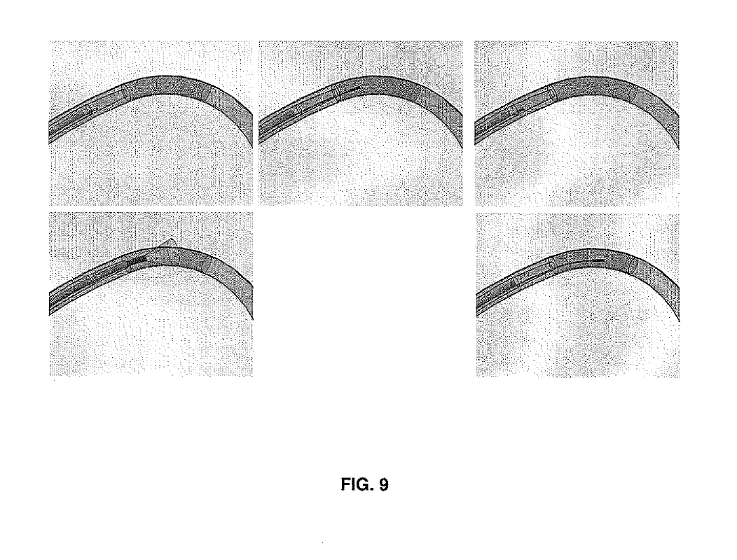

FIG. 9 shows one embodiment of the present invention as applied to the navigation of a forward-imaging catheter and ablating device through a CTO. The forward-imaging modality can be ultrasound or infrared/optical imaging with optical coherence tomography, for instance. A multi-lumen guide catheter or advancement catheter has been navigated proximal to the occlusion. An RF ablation wire is then advanced and ablates through part of the lesion; then the imaging catheter is advanced through the lesion bore created by the RF device, and provides imaging data and characterization data that enable determination of the next RF wire navigation and ablation steps. This process is repeated in as many steps as are necessary to effect lesion crossing. In an alternate embodiment, the multi-lumen catheter itself is advanced through the lesion as the RF ablation wire progresses through the lesion material. In an alternate embodiment, a crossing wire that can apply a sufficient level of mechanical push force can be used to cross the occlusion. An example of such a device is a magnetic guidewire that also incorporates a coil made of a paramagnetic material, such as Platinum-Cobalt alloy; such wires can withstand relatively large mechanical forces before buckling.

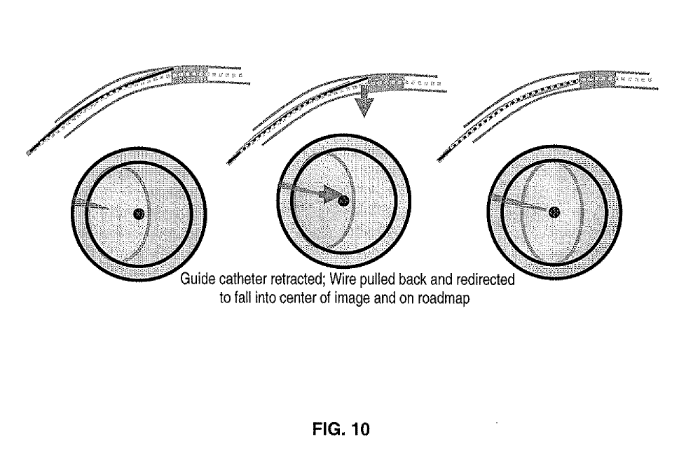

FIG. 10 illustrates the use of side-looking imaging technology to the progression of the therapeutic device through the CTO. The method includes the steps of importing a pre-operative 3D data set, such as acquired by a CT or MRI or MRA imaging system; defining the road map, in dashed lines in the figure, and advancing an imaging catheter and wire proximal to the lesion; further advancing the imaging catheter and wire together through the lesion proximal end while monitoring on live fluoroscopy the progress of the intervention; registering the real-time imaging catheter images to the cross-sectional image data (or conversely), noting the vessel local eccentricity and other characteristic features; retracting the catheter, deflecting the ablation wire to reorient its tip with respect to the road map or until it is essentially in coincidence with the lumen center as determined from imaging. This process is enabled with an imaging catheter that is approximately located at the catheter tip.

As will be further described below, the crossing wire can be used with the imaging catheter to navigate through the occlusion in a variety of embodiments, after suitable registration.

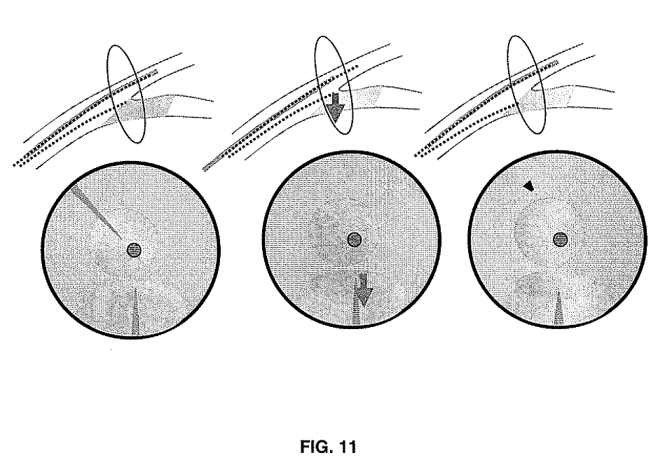

In one embodiment illustrated in FIG. 11, a side-looking imaging catheter that uses either ultrasound or infrared/optical imaging to image the lumen or lumen wall can be positioned distal to a branch, or in a parallel vessel to the occluded vessel. The crossing wire is then advanced to the proximal end of the lesion in the occluded vessel. The imaging catheter in the parallel vessel or branch may need to be adjusted, such that the crossing wire, and the lumen of the occluded vessel, is visible in the imaging plane/field of view of the imaging catheter. If, in the real-time image, the wire is seen to be close to the lumen wall (media/adventitia tissue interface) of the occluded vessel, the remote navigation system is used to steer the crossing wire away from the vessel wall and toward the center of the lumen. For instance, in the case of a magnetic navigation system, a suitable magnetic field is applied causing the (magnetically endowed) crossing wire to orient itself away from the lumen wall. The crossing wire is then adjusted to allow proper re-orientation and then advanced.

The crossing wire can be a wire with sufficient mechanical stiffness to enable pushing through the occlusion, or it can be an ablative device that uses, for instance, RF energy or laser energy to actively create an opening in the occluded portion. In one embodiment, it can mechanically deliver ultrasonic pulses that act as a local “jackhammer” to chip away at the lesion. In the case of active energy delivery, after the wire is adjusted and re-oriented energy is suitably delivered to create an opening for the wire to be locally advanced in the lesion. The process of real-time imaging, reorienting actuation, energy delivery and wire advancement is iteratively repeated as needed to completely cross the occlusion. The imaging catheter in the branch vessel or parallel vessel may need to be suitably repositioned so that the crossing wire remains in the field of view of the imaging catheter.

After the occlusion has been crossed with the wire, a therapeutic device such as a stent delivery device is advanced over the wire and positioned within the lesion. The stent is expanded and delivered in place to hold the vessel open in the area of the lesion. In one embodiment of the methods of this invention, the therapeutic device closely follows the crossing wire as it is advanced through the lesion. In some cases, the crossing wire can be precessed locally as it is advanced or retracted together with simultaneous energy delivery (RF or laser ablation) in order to locally enlarge the opening created, so that the therapeutic device can be easily advanced through the opening.

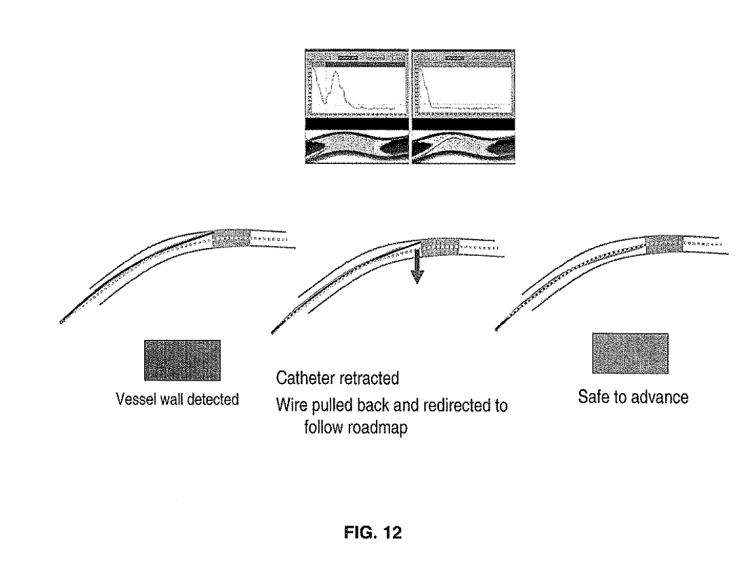

In an alternate embodiment described in FIG. 12, the imaging catheter can be a wall sensing device that either closely follows, or is integrated with, the crossing wire. In one example of this embodiment, the sensing catheter can use Optical Coherence Reflectometry (OCR) to estimate the nearest distance from the catheter to the vessel wail, and this information can be used to indicate whether the catheter is in a safe zone (far enough away from the wall) or in an unsafe zone (too close to the vessel wall) with respect to the vessel wall. In another embodiment, instead of just two zones, a larger number of zones could be indicated.

The crossing wire tip would be located close to the imaging catheter tip, and if the device is determined to be close to the vessel wall, it would be steered away from the wall by the remote navigation system. Ablative energy is delivered for creating an opening in the lesion, and the process of sensing wall proximity, device re-orienting, energy delivery and device advancement is iteratively repeated to cross the lesion.

In another preferred embodiment, and referring now again to FIG. 9, a forward-looking imaging catheter within the same vessel as the crossing wire is used to image the location of the vessel wall in front of the device. This image information can be used to determine the steering of the crossing wire while stepping forward through the occlusion. In this embodiment, the real-time image data is obtained as a conical region ahead of the imaging catheter. If the image is displayed as a circular image, it is helpful therefore, to indicate a distance scale, such that points at a larger radius from the center are also at a larger axial distance; the distance scale could be a color bar or a line with a set of markings indicating forward axial location. In one embodiment, the imaging catheter and the crossing wire are both carried within a guide catheter and the devices can be (in alternating fashion) advanced and retracted relative to each other. If the blood vessel is large enough to permit simultaneous passage of both imaging catheter and crossing wire, the wire itself (and its shadow) can be seen within the real-time image when the wire is in front of the imaging catheter. In this case since the wire is directly visualized, it can be appropriately steered away from the (forward) vessel wall. If there is insufficient room within the vessel to permit simultaneous passage of both catheter and wire devices, the real-time image is used to note the desired (“safe”) steering direction for the wire, and a suitable wire re-orientation is applied as the crossing wire is advanced and ablation energy delivered. This process is iteratively continued. In some cases, it may be necessary to precess the wire in order to enlarge the opening created by the wire, so that the imaging catheter and other therapeutic devices can also follow the wire to cross the occlusion.

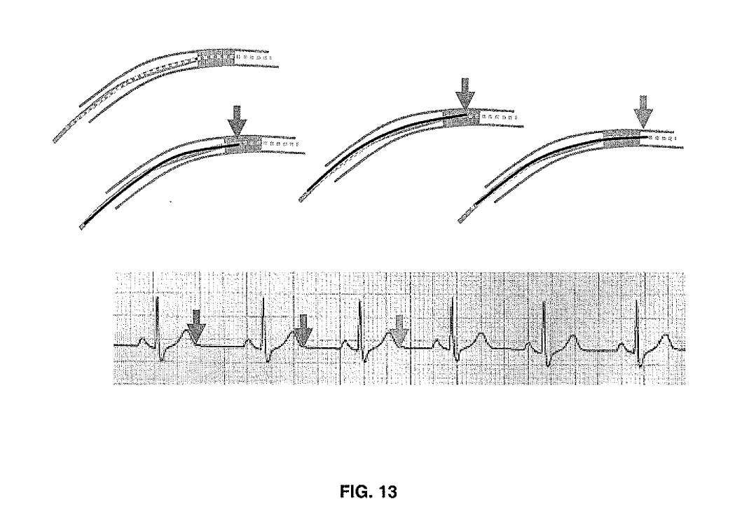

As is illustrated in FIG. 13, it is desirable to gate the delivery of RF energy to the cardiac cycle as determined from an ECG measurement, and also to the respiratory cycle. It is known that the heart is most sensitive to electrical stimulation during the ST segment of the cycle. Also, RF energy deposition triggering can trigger systole.

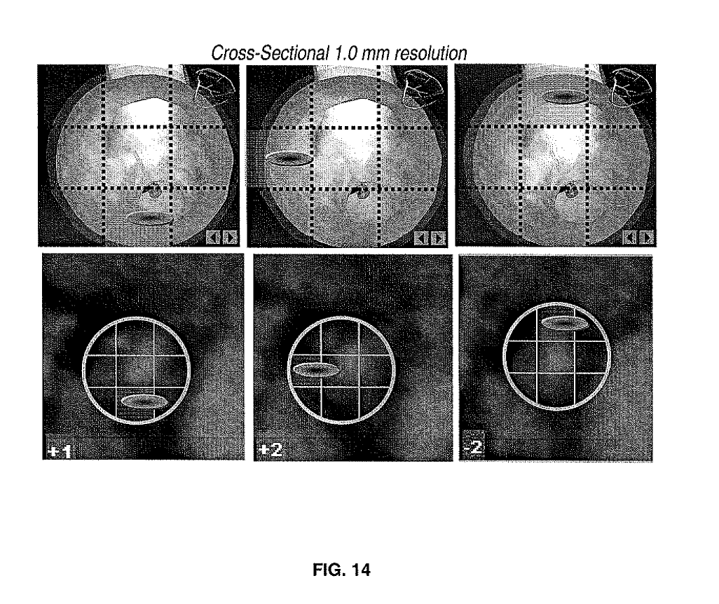

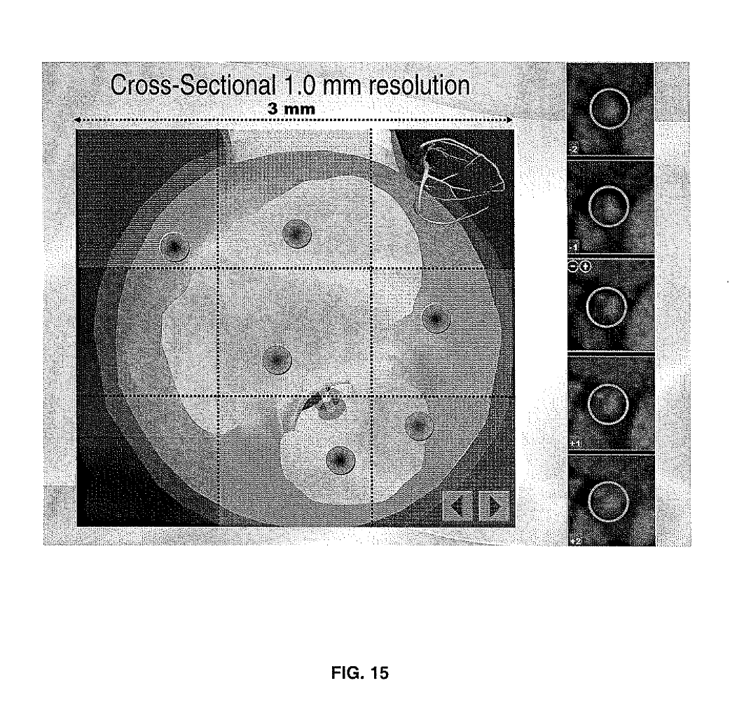

FIGS. 14 and 15 illustrate the use of wire localization technology to navigation using an endo-luminal view. Recently, and as known in the art, technologies have evolved to the point that it is possible to mount a GPS-like element on the tip or along a guidewire; the element dimensions are small enough so as not to impede the wire progression through anatomy, and position and localization information can be as accurate as 0.5-1 mm. If the wire position can be automatically determined and localization known to within about 1 mm, resolution is sufficient to enable micro-navigation using an endo-luminal view. Accordingly, the wire position with respect to the vessel center can be biased to bypass a calcified lesion and realign with the effective lumen center. FIGS. 14 and 15 shows the use of a navigation vector rendition superposed onto the endoluminal views; and the corresponding cross-sectional images available for example from a pre-operative imaging study.

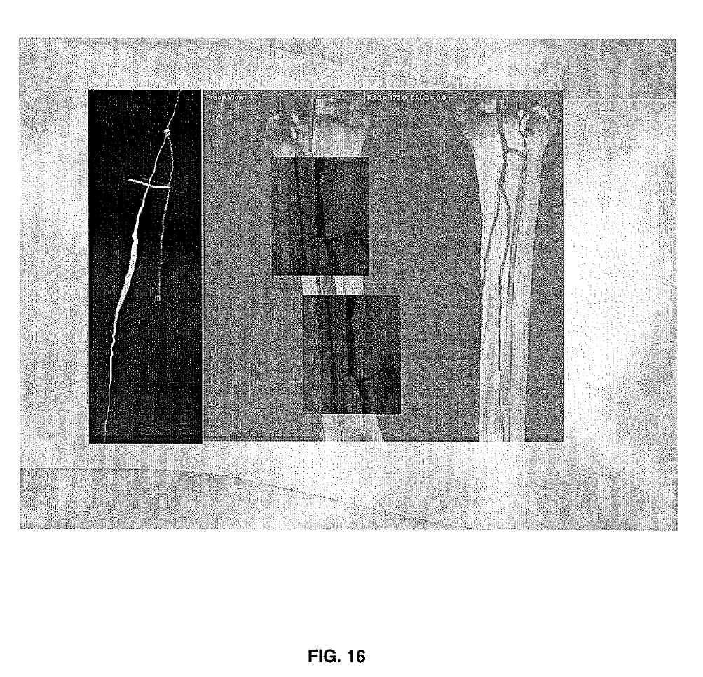

In another aspect of some embodiments of the invention, FIG. 16 illustrates the use of a sliding x-ray projection to the mapping and navigation of elongated vasculature, such as encountered for example when imaging legs and extremities.

It is appreciated that in the clinical application of RF ablation, the amount of RF power applied to the lesion sufficient to effect advancement and crossing varies greatly from one lesion to another, and even within a given lesion, from one point to another. For instance, a number of CTO are known to present a fibrous cap that is more difficult to ablate than lipid plaque contents. Sometimes increased RF power is required only to penetrate through such a cap, while progression through the remainder of the lesion requires significantly less power. Other lesions present extended calcifications throughout and much increased power (as compared to that needed for lipid tissue) is required to advance the RF wire through and cross the lesion.

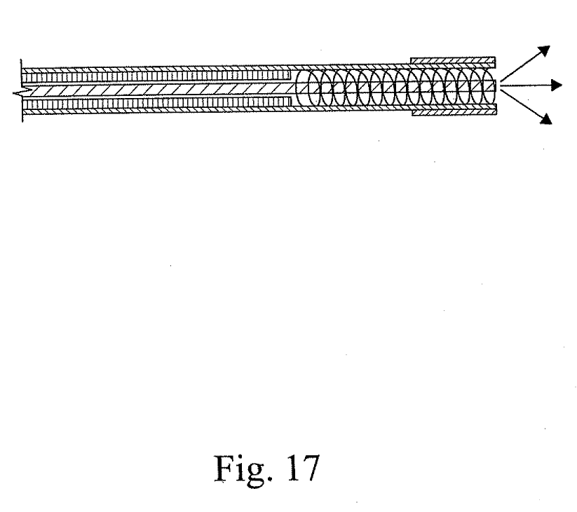

FIG. 17 presents one embodiment of an RF-ablation and optical imaging or sensing combination device designed according to the principles of the present invention to facilitate complex occlusions and CTO crossing. It is understood that many variations can be made to the design specifics by those skilled in the art. In FIG. 17, an optical fiber is provided from the device proximal end to the distal end that can carry one or more optical frequencies, or frequency bands, for imaging or characterization of the tissues. In the device of the figure, a single fiber optic is provided, and the fiber is polished at the distal end or other wise a lens is mounted at the fiber optic distal end (not shown) to shine the light into the tissue and detect the reflected and or scattered light. In other embodiments, two or more fiber optic channels are provided. Further, in the embodiment of the figure, a bi-polar RF assembly is provided to carry RF energy to the distal tip and into the tissues, and provide a return path. In other embodiment, a unipolar design can be adopted. In the figure, a distal coil is shown, for example made of stainless steel, or in magnetic navigation applications made of a material, such as platinum cobalt (PtCo) providing a residual magnetization Br of about 0.6 T; alternatively PtFeNb could provide a Br of about 1.0 T. Alternatively or in addition to the coil design, the distal end could comprise a hollow magnet.

Within an integrated device comprising a multi-lumen elongated body, one lumen or channel could be designed to permit advancement over a guidewire; a second lumen could accommodate an optical component, or alternatively carry lead for an IVUS element located near the device distal end; and a third lumen could be designed to carry RF energy to a an RF-ablative electrode located at or near the device distal end. Alternatively, one or more of the integrated device lumen could provide passage way for separately extendable device(s), that could be navigated from the integrated device distal end to the vessel of interest or lesion to be treated.

Although the method has been illustrated for magnetic navigation applications, it is clear that it may also be applied in conjunction with other means of navigation. For example, the navigation means may comprise mechanical actuation, as per use of a set of pull-wires that enable distal device bending, by itself or in conjunction with proximal device advance and rotation. The navigation means may also comprise other techniques known in the art, such as electrostrictive device control. Further navigation means may comprise combination of the above methods, such as combination of magnetic and electrostrictive navigation, combination of mechanical and electrostrictive navigation, or combination of magnetic and mechanical navigation.

The advantages of the above described embodiments and improvements should be readily apparent to one skilled in the art, as to enabling CTO and occlusive lesion crossing therapy. Additional design considerations, or a variety of technologies, such as various lesion crossing/opening technologies and different imaging modalities, may be incorporated without departing from the spirit and scope of the invention. Accordingly, it is not intended that the invention be limited by the particular embodiment or form described above, but only by the appended claims.

1. (canceled)

2. A method of crossing an occlusive vascular lesion with a remotely actuated interventional device, comprising:
(i) remotely steering the device to an occlusion;
(ii) performing local vessel characterization with an accessory local characterization device;
(iii) re-orienting the interventional device by remote actuation based on the local characterization;
(iv) applying mechanical push to the device to push into the occlusion;
(v) adjusting the longitudinal location of the device relative to the vessel; and
(vi) iterating through steps (ii) to (v) to cross the occlusion.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. The method of claim 2, wherein the remote actuation comprises mechanical actuation of the device distal end.

16. The method of claim 2, wherein the remote actuation comprises electrostrictive actuation of the device distal end.

17. The method of claim 2, wherein the interventional device is advanced through a guide catheter.

18. The method of claim 2, wherein the occlusive lesion is a chronic total occlusion.

19. The method of claim 2, wherein the characterization is performed as local imaging with ultrasound.

20. The method of claim 2, wherein the characterization is performed with optical coherence reflectometry.

21. The method of claim 2, wherein the characterization is performed as local imaging with optical coherence tomography.

22. The method of claim 2, wherein the characterization is performed as local wall sensing with optical coherence reflectometry.

23. A magnetic navigation system for crossing occlusive lesions, comprising:
means for controllably and remotely navigating an interventional medical device within a subject's lumen to an occlusion;
means for characterizing tissues in the vicinity of the interventional device distal end;
means for positioning and orienting the interventional device distal end with respect to the occlusion diseased tissues to be ablated; and
means for applying ablative energy to the occlusion diseased tissues.

24. The magnetic navigation system of claim 23, further comprising additional navigation means selected from the group consisting of
i) mechanical navigation means;
ii) electrostrictive navigation means; and
iii) hydraulic navigation means.

25. The magnetic navigation system of claim 23, wherein the ablative energy is derived from a radio-frequency ablation means.

26. A method of navigating an integrated device for the treatment of a lesion with a remote navigation system, the method comprising:
providing an integrated device with means for tissue characterization and means for tissue opening creation;
navigating the distal tip of the integrated device to the vicinity of the lesion;
iteratively characterizing tissues in the vicinity of the ablative device distal end, creating an opening through tissue, and advancing at least part of the integrated device through the created opening; and
whereby the lesion is crossed by at least part of the integrated device.

27. The method of claim 26, wherein tissue characterization is performed by optical coherence reflectometry.

28. The method of claim 26, wherein tissue characterization is performed by near infrared diffuse reflectance spectroscopy.

29. The method of claim 26, wherein the creation of a tissue opening is performed by an RF ablation device.

30. The method of claim 26, wherein the creation of a tissue opening is performed by a laser ablation device.

31. (canceled)

32. (canceled)

33. (canceled)

34. (canceled)

35. (canceled)

36. (canceled)

37. (canceled)

38. (canceled)

39. (canceled)

40. (canceled)

41. (cancelled)

42. (canceled)

43. A method of crossing an occlusive vascular lesion with an interventional device remotely actuated by a remote navigation system, comprising:
(i) remotely steering the device to an occlusion;
(ii) performing local vessel characterization with an accessory local characterization device;
(iii) performing spatial registration of the local characterization data to the remote navigation system;
(iv) re-orienting the interventional device by remote actuation based on the current local characterization;
(v) applying ablative RF energy to the occlusion;
(vi) adjusting the longitudinal location of the device relative to the vessel; and
(vii) iterating through steps (iii) to (vi) to cross the occlusion.
